(19) United States
(12) Patent Application Publication
(10) Pub. No.: US 2008/0234556 A1
(43) Pub. Date: Sep. 25, 2008

(54) METHOD AND APPARATUS FOR SENSING RESPIRATORY ACTIVITIES USING SENSOR IN LYMPHATIC SYSTEM

(75) Inventors: M. Jason Brooke, Minneapolis, MN (US); Yachuan Pu, Minneapolis, MN (US); Allan C. Shuros, St. Paul, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc., St. Paul, MN (US)

(21) Appl. No.: 11/688,380

(22) Filed: Mar. 20, 2007

(51) Int. Cl. A61B 5/08; A61N 1/36; A61N 1/362

(52) U.S. Cl. 600/301; 600/529; 607/2; 607/9

(57) ABSTRACT

A respiratory monitoring system includes an implantable lymphatic sensor configured to be placed in a lymphatic vessel, such as the thoracic duct or a vessel branching from the thoracic duct, near the diaphragm. The implantable lymphatic sensor senses a signal indicative of respiratory activities. Examples of the signal include a diaphragmatic activity signal indicative of movement of the diaphragm and a transthoracic impedance signal indicative of pulmonary volume. In one embodiment, the respiratory monitoring system is incorporated into a cardiac rhythm management system that controls cardiac therapy delivery using the signal sensed by the implantable lymphatic sensor.

### TECHNICAL FIELD

This document relates generally to medical devices and particularly to an implantable system that senses respiratory activities using a sensor placed in a lymphatic vessel.

### BACKGROUND

The heart is the center of a person's circulatory system. It includes an electro-mechanical system performing two major pumping functions. The left portions of the heart draw oxygenated blood from the lungs and pump it to the organs of the body to provide the organs with their metabolic needs for oxygen. The right portions of the heart draw deoxygenated blood from the organs and pump it into the lungs where the blood gets oxygenated. The pumping functions are accomplished by contractions of the myocardium (heart muscles). In a normal heart, the sinoatrial node, the heart's natural pacemaker, generates electrical impulses, known as action potentials, that propagate through an electrical conduction system to various regions of the heart to excite myocardial tissues in these regions. Coordinated delays in the propagations of the action potentials in a normal electrical conduction system cause the various regions of the heart to contract in synchrony such that the pumping functions are performed efficiently.

A blocked or otherwise damaged electrical conduction system causes irregular contractions of the myocardium, a condition generally known as arrhythmia. Arrhythmia reduces the heart's pumping efficiency and hence, diminishes the blood flow to the body. A deteriorated myocardium has decreased contractility, also resulting in diminished blood flow. A heart failure patient usually suffers from both a damaged electrical conduction system and a deteriorated myocardium. The diminished blood flow results in insufficient blood supply to various body organs, preventing these organs from functioning properly and causing various symptoms.

Implantable cardiac rhythm management (CRM) devices, such as cardiac pacemakers and cardioverter/defibrillators, are used to treat various cardiac disorders including cardiac arrhythmias and heart failure. Respiratory activities are monitored in implantable CRM devices for various purposes. A respiratory signal may be sensed for use as an input in closed-loop system that controls of the delivery of a cardiac therapy. This provides for, for example, coordination of cardiac and respiratory activities that allow control of the delivery of the cardiac therapy for efficient blood oxygenation and circulation. It has been observed that synchronization of delivery of pacing or defibrillation therapy to respiratory cycles may reduce the energy required for an effective delivery of the therapy. Various cardiopulmonary conditions or disorders may be detected using respiratory parameters extracted from the respiratory signal. Such cardiopulmonary conditions or disorders indicate a need to start, stop, or adjust the delivery of a cardiac therapy. For example, heart failure is known to be associated with respiratory disorders such as sleep disordered breathing. Detection of such respiratory disorders allows prevention of a heart failure therapy from affecting the patient's respiratory functions to an intolerable or unacceptable extent. A sensed respiratory signal may also be used to minimize effects of respiratory activities in cardiac signal sensing and parameter measurements. For these and other reasons, there is a need for sensing respiratory activities in an implantable CRM system.

### SUMMARY

A respiratory monitoring system includes an implantable lymphatic sensor configured to be placed in a lymphatic vessel, such as the thoracic duct or a vessel branching from the thoracic duct, near the diaphragm. The implantable lymphatic sensor senses a signal indicative of respiratory activities. Examples of the signal include a diaphragmatic activity signal indicative of movement of the diaphragm and a transthoracic impedance signal indicative of pulmonary volume.

In one embodiment, a system includes a lymphatic sensor assembly and a sensor signal processor. The lymphatic sensor assembly is configured to be implanted in a lymphatic vessel and includes a lymphatic sensor connected to a lymphatic sensor base. The lymphatic sensor senses a lymphatic sensor signal indicative of respiratory activities. The lymphatic sensor base allows the lymphatic sensor assembly to be implanted in the lymphatic vessel. The sensor signal processor is communicatively coupled to the lymphatic sensor and includes a sensor signal input, a respiration monitor, and a respiratory parameter generator. The sensor signal input receives the lymphatic sensor signal The respiration monitor produces a respiratory signal indicative of the respiratory activities by processing the lymphatic sensor signal. The respiratory parameter generator produces one or more respiratory parameters using the respiratory signal.

In one embodiment, a method for monitoring respiratory activities is provided. A lymphatic sensor signal is sensed using a lymphatic sensor implanted in a lymphatic vessel. The lymphatic sensor signal is indicative of respiratory activities. A respiratory signal indicative of the respiratory activities is produced using the lymphatic sensor signal. One or more respiratory parameters are produced using the respiratory signal.

This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is an illustration of an embodiment of a respiratory monitoring system and portions of an environment in which the respiratory monitoring system is used.

FIG. 2 is an illustration of another embodiment of the respiratory monitoring system and portions of the environment in which the respiratory monitoring system is used.

FIG. 3 is a block diagram illustrating an embodiment of portions of the respiratory monitoring system.

FIG. 4 is a block diagram illustrating an embodiment of a sensor signal processor of the respiratory monitoring system.

FIG. 5 is an illustration of an embodiment of a lymphatic sensor assembly of the respiratory monitoring system.

FIG. 6 is a block diagram illustrating an embodiment of a CRM system including the respiratory monitoring system.

FIG. 7 is a block diagram illustrating an embodiment of an implantable medical device of the CRM system.

FIG. 8 is a block diagram illustrating an embodiment of the external system of the CRM system.

FIG. 9 is a flow chart illustrating a method for monitoring respiratory activities using a sensor placed in a lymphatic vessel.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

Respiratory activities are monitored by implantable CRM devices to provide closed-loop control for adjusting cardiac therapy parameters. For example, a known pacing system senses a transthoracic impedance signal using an electrode incorporated into a subcutaneously implanted pacemaker and another electrode incorporated into a distal end of a pacing lead for intracardiac placement. A respiratory signal, which is extracted from the transthoracic impedance signal by removing noise including cardiac electrical signals, represents lung volume. The present system monitors respiratory activities using a lymphatic sensor implanted within a lymphatic vessel, such as the thoracic duct or a vessel branching from the thoracic duct, near the diaphragm. The lymphatic sensor senses an activity signal indicative of movement of the diaphragm or an impedance signal representative of a substantial portion of the lung, thereby providing a higher signal-to-noise ratio and/or a better representation of the lung volume than the know pacing system.

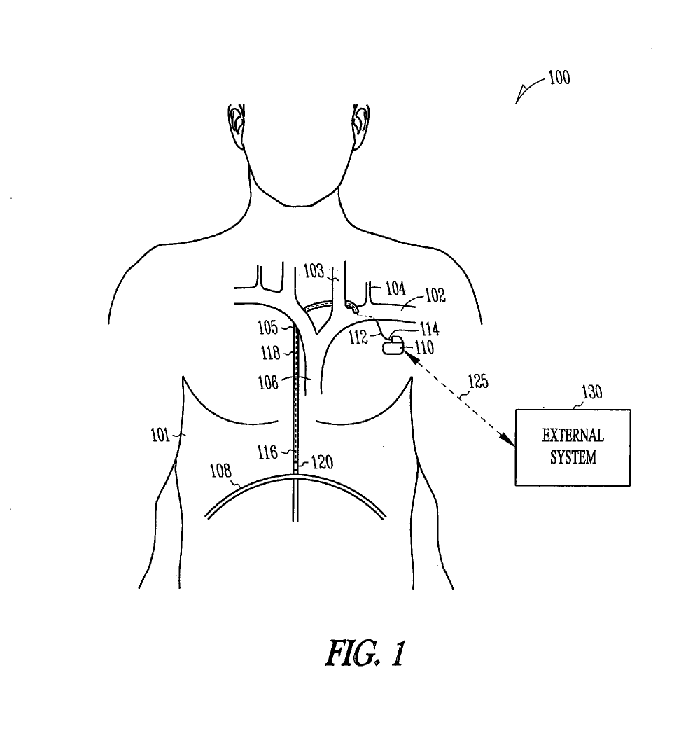

FIG. 1 is an illustration of an embodiment of a respiratory monitoring system 100 and portions of an environment in which system 100 is used. System 100 includes an implantable medical device 110, a lead 112, a lymphatic sensor assembly 120 incorporated into lead 112, an external system 130, and a telemetry link 125 providing for communication between implantable medical device 110 and external system 130.

System 100 monitors respiratory activities via a thoracic duct 105, which is part of the lymphatic system of a patient's body 101. The lymphatic system includes lymph tissue, nodes, and vessels. Interstitial fluid is absorbed from tissue, filtered through lymph nodes, and empties into lymphatic vessels. FIG. 1 illustrates portions of thoracic duct 105, a subclavian vein 102, a left external jugular vein 104, a left internal jugular vein 103, and a superior vena cava 106. Thoracic duct 105 connects to the venous system at the juncture of subclavian vein 102 and a left internal jugular vein 103. The fluid (lymph) from the lower body flows up to thoracic duct 105 and empties into subclavian vein 102 from thoracic duct 105. Thoracic duct 105 is located in the posterior mediastinal area of body 101, adjacent to the heart and various portions of the nervous system including portions of the vagus, sympathetic, and phrenic nerves. In the illustrated embodiment, thoracic duct 105 is used as a conduit for advancing the sensor to a location suitable for sensing a signal indicative of respiratory activities. This approach to the process of sensor placement has the potential advantage of reducing invasiveness of implantation procedure under many circumstances.

Implantable medical device 110 processes the signal indicative of respiratory activities. In various embodiments, implantable medical device 110 is also capable of sensing other physiological signals and/or delivering therapies in addition to the respiratory activity monitoring. Examples of such additional therapies include cardiac pacing therapy, cardioversion/defibrillation therapy, cardiac resynchronization therapy (CRT), cardiac remodeling control therapy (RCT), drug therapy, cell therapy, and gene therapy. In various embodiments, implantable medical device 110 monitors respiratory activities for used in a closed-loop system that controls the delivery of one or more of such additional therapies. In one embodiment, in addition to lead 112, system 100 includes one or more endocardial and/or epicardial leads connected to implantable medical device 110 for delivering pacing and/or defibrillation pulses to the heart.

Lead 112 is an implantable lead including a proximal end 114, a distal end 116, and an elongate lead body 118 between proximal end 114 and distal end 116. Proximal end 114 is configured to be connected to implantable medical device 110. Lead 112 includes lymphatic sensor assembly 120. Lymphatic sensor assembly 120 includes a lymphatic sensor for sensing the signal indicative of respiratory activities from a location in a lymphatic vessel such as thoracic duct 105 or a location accessible through the lymphatic vessel. In one embodiment, the lymphatic sensor includes an activity sensor that senses a diaphragmatic activity signal indicative of movement of a diaphragm 108 of body 101. In another embodiment, the lymphatic sensor includes an impedance sensor that senses a transthoracic impedance signal indicative of pulmonary volume of body 101. In the illustrated embodiment, lymphatic sensor assembly 120 is incorporated into distal end 116 for placement near diaphragm 108. In another embodiment, lymphatic sensor assembly 120 is incorporated into a distal portion of elongate lead body 118, away from distal end 116, for placement near diaphragm 108. In one embodiment, distal end 116 also includes one or more sensing and/or stimulation electrodes, such as those discussed in U.S. patent application Ser. No. 11/422,421, entitled “METHOD AND APPARATUS FOR NEURAL STIMULATION VIA THE LYMPHATIC SYSTEM,” filed on Jun. 6, 2006, assigned to Cardiac Pacemakers, Inc., which is incorporated herein by reference in its entirety. In one embodiment, lead 112 is configured to allow drug delivery. In a specific embodiment, implantable medical device 110 includes a drug pump, and lead 112 includes a lumen with one end configured to be connected to the drug pump and other one or more ends each connected to a drug delivery port along elongate lead body 118 and/or at distal end 116. In another embodiment, one or more drug delivery devices, such as polymeric drug collars, are incorporated into lead 112 along elongate lead body 118 and/or at distal end 116.

The distal portion of elongate lead body 118 (a substantial portion of elongate lead body 118 coupled to distal end 116) is configured for placement in subclavian vein 102 and thoracic duct 105, such that distal end 116 is placed in thoracic duct 105. During the implantation of lead 112, distal end 116 is inserted into subclavian vein 102 through an incision, advanced in subclavian vein 102 toward thoracic duct 105, inserted into thoracic duct 105 from subclavian vein 102, and advanced in thoracic duct 105 until the lymphatic sensor reaches a location near diaphragm 108. In one embodiment, the position of distal end 116 is adjusted by monitoring the quality of the signal sensed by the lymphatic sensor. In one embodiment, lymphatic sensor assembly 120 includes a fixation mechanism configured to stabilize the lymphatic sensor in a position in thoracic duct 105. One example of method and apparatus for accessing the lymphatic system is discussed in U.S. patent application Ser. No. 11/422,423, entitled “METHOD AND APPARATUS FOR LYMPHATIC SYSTEM PACING AND SENSING,” filed on Jun. 6, 2006, assigned to Cardiac Pacemakers, Inc., which is incorporated herein by reference in its entirety.

In one embodiment, lead 112 is configured to allow distal end 116 to be further advanced into a vessel branching from thoracic duct 105 such that lymphatic sensor assembly 120 can be placed in a location providing for better respiratory activity sensing, if available. After distal end 116 is inserted into thoracic duct 105, it is advanced to the junction of thoracic duct 105 and the branching vessel and inserted to the branching vessel.

External system 130 communicates with implantable medical device 110 and provides for access to implantable medical device 110 by a physician or other caregiver. In one embodiment, external system 130 includes a programmer. In another embodiment, external system 130 is a patient management system including an external device communicating with implantable medical device 110 via telemetry link 125, a remote device in a relatively distant location, and a telecommunication network linking the external device and the remote device. The patient management system allows access to implantable medical device 10 from a remote location, for purposes such as monitoring patient status and adjusting therapies. In one embodiment, telemetry link 125 is an inductive telemetry link. In another embodiment, telemetry link 125 is a far-field radio-frequency (RF) telemetry link. Telemetry link 125 provides for data transmission from implantable medical device 110 to external system 130. This includes, for example, transmitting real-time physiological data acquired by implantable medical device 110, extracting physiological data acquired by and stored in implantable medical device 110, extracting patient history data such as occurrences of predetermined types of pathological events and therapy deliveries recorded in implantable medical device 110, and/or extracting data indicating an operational status of implantable medical device 110 (e.g., battery status and lead impedance). Telemetry link 125 also provides for data transmission from external system 130 to implantable medical device 110. This includes, for example, programming implantable medical device 110 to acquire physiological data, programming implantable medical device 110 to perform at least one self-diagnostic test (such as for a device operational status), and/or programming implantable medical device 110 to deliver one or more therapies and/or to adjust the delivery of one or more therapies.

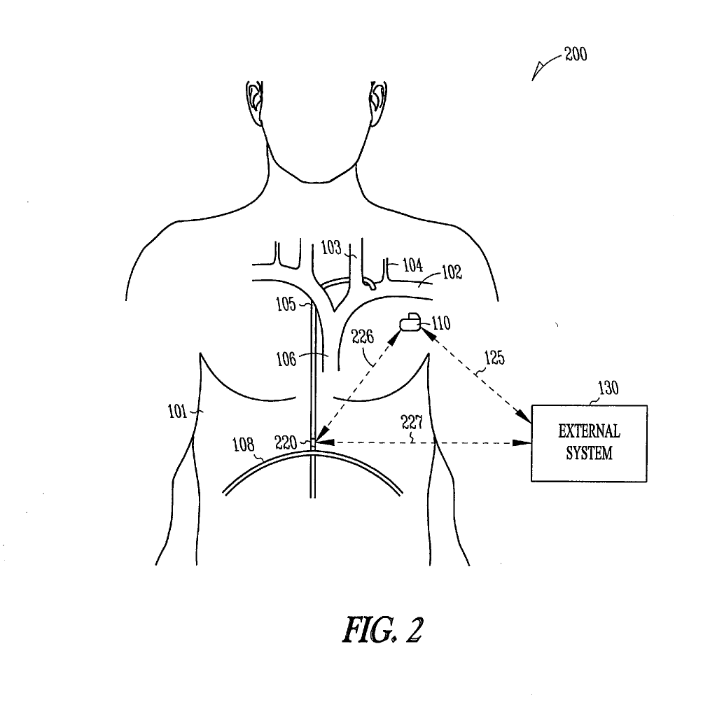

FIG. 2 is an illustration of an embodiment of a respiratory monitoring system 200 and portions of the environment in which system 200 is used. As illustrated, system 200 includes an implantable medical device 110, a lymphatic sensor assembly 220, an external system 130, a telemetry link 125 providing for communication between implantable medical device 110 and external system 130, a telemetry link 226 providing for communication between implantable medical device 110 and lymphatic sensor assembly 220, and a telemetry link 227 providing for communication between lymphatic sensor assembly 220 and external system 130. Lymphatic sensor assembly 220 includes the lymphatic sensor for sensing the signal indicative of respiratory activities. System 200 is substantially identical to system 100 except that the lymphatic sensor is wirelessly coupled to implantable medical device 110.

In one embodiment, lymphatic sensor assembly 220 is delivered using a percutaneous transluminal catheter in a manner similar to the insertion of lead 112. During the implantation, lymphatic sensor assembly 220 is attached to the distal end of the catheter and inserted into subclavian vein 102 through an incision, advanced in subclavian vein 102 toward thoracic duct 105, inserted into thoracic duct 105 from subclavian vein 102, and advanced in thoracic duct 105 until the lymphatic sensor reaches a location near diaphragm 108. In one embodiment, the position of lymphatic sensor assembly 220 is adjusted by monitoring the quality of the signal sensed by the lymphatic sensor. Lymphatic sensor assembly 220 includes a fixation mechanism configured to stabilize lymphatic sensor assembly 220 in thoracic duct 105. After the positioning of lymphatic sensor assembly 220 is completed, the fixation mechanism is deployed, and the catheter is withdrawn from body 101. Implantable medical device 110 communicates with the lymphatic sensor via telemetry link 226.

In one embodiment, lymphatic sensor assembly 220 is configured to be further advanced into a vessel branching from thoracic duct 105 such that lymphatic sensor assembly 220 can be placed in a location providing for better respiratory activity sensing, if available. After lymphatic sensor assembly 220 is inserted into thoracic duct 105, it is advanced to the junction of thoracic duct 105 and the branching vessel and inserted to the branching vessel.

While the placement of the lymphatic activity sensor in the thoracic duct, or a vessel branching from the thoracic duct, is specifically discussed, with reference to FIGS. 1 and 2, as an example for providing for a suitable location for sensing respiratory activity, the present subject matter generally includes introducing one or more sensors to a desirable sensing site via a lymphatic vessel. In various embodiments, respiratory activities are sensed using one or more sensors placed in a lymphatic vessel and/or one or more sensors placed in a structure that is accessible through the lymphatic vessel, including another lymphatic vessel, or a vein, branching from the lymphatic vessel.

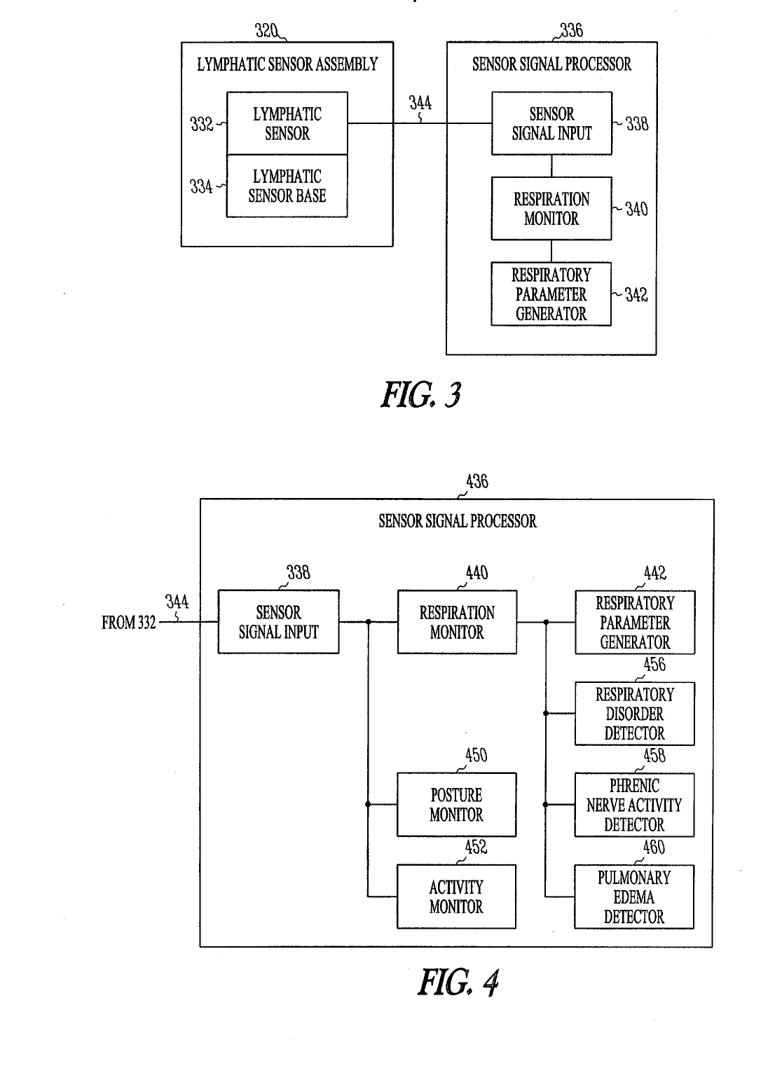

FIG. 3 is a block diagram illustrating an embodiment of portions of respiratory monitoring system 100 or 200, including a lymphatic sensor assembly 320 and a sensor signal processor 336.

Lymphatic sensor assembly 320 represents an embodiment of lymphatic sensor assembly 120 or 220 and includes a lymphatic sensor 332 and a lymphatic sensor base 334. Lymphatic sensor 332 senses a lymphatic sensor signal indicative of respiratory activities. In one embodiment, lymphatic sensor 332 includes a diaphragmatic activity sensor that senses a diaphragmatic activity signal indicative of movement of diaphragm 108. The movement of diaphragm 108 results in the respiratory activities. In a specific embodiment, the diaphragmatic activity sensor is an accelerometer that senses an accelerometer signal indicative of the movement of diaphragm 108. In various specific embodiments, the accelerometer signal is also used as an activity signal indicative of the level gross physical activity of body 101 and/or a posture signal indicative of posture of body 101. In another specific embodiment, the diaphragmatic activity sensor is a strain gauge that senses a strain gauge signal indicative of the movement of diaphragm 108. In another specific embodiment, the diaphragmatic activity sensor is a piezoelectric sensor that senses a piezoelectric sensor signal indicative of the movement of diaphragm 108. In another embodiment, lymphatic sensor 332 includes an impedance sensing electrode for sensing a transthoracic impedance signal indicative of pulmonary volume. In a specific embodiment, the transthoracic impedance signal is sensed using lymphatic sensor 332 (electrode) and another electrode incorporated onto implantable medical device 110. Such an electrode configuration allows sensing of a transthoracic impedance signal that provides for a better representation of the pulmonary volume than an electrode configuration using an intracardiac electrode and the electrode incorporated onto implantable medical device 110. Lymphatic sensor base 334 is connected to lymphatic sensor 332 and configured to allow lymphatic sensor assembly 320 to be stabilized in a lymphatic vessel such as thoracic duct 105. An example of lymphatic sensor base 334 is discussed below, with reference to FIG. 5.

Sensor signal processor 336 is communicatively coupled to lymphatic sensor 332 via a communication link 344, which is a wired link or a wireless telemetry link. Sensor signal processor 336 includes a sensor signal input 338, a respiration monitor 340, and a respiratory parameter generator 342. Sensor signal input 338 receives the lymphatic sensor signal sensed by lymphatic sensor 332. Respiration monitor 340 produces a respiratory signal indicative of the respiratory activities by processing the lymphatic sensor signal. Respiratory parameter generator 342 produces one or more respiratory parameters using the respiratory signal.

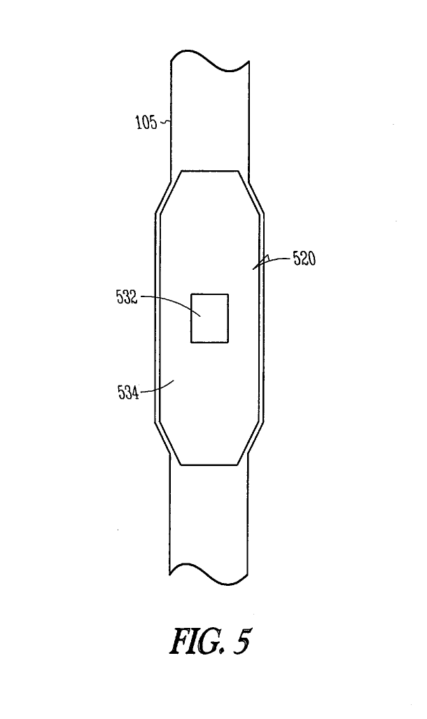

FIG. 4 is a block diagram illustrating an embodiment of a sensor signal processor 436, which represents a specific embodiment of sensor signal processor 336. Sensor signal processor 436 includes sensor signal input 338, a respiration monitor 440, a respiratory parameter generator 442, a respiratory disorder detector 456, a phrenic nerve activity detector 458, a pulmonary edema detector 460, a posture monitor 450, and an activity monitor 452.

Respiration monitor 440 represents a specific embodiment of respiratory monitor 340 and receives the lymphatic sensor signal and produces a respiratory signal indicative of the respiratory activities using the lymphatic sensor signal. Respiration monitor 440 includes a band-pass filter to remove noise associated with non-respiratory activities, such as the gross physical activity of body 101. In one embodiment, the band-pass filter for producing the respiratory signal has a high-pass cutoff frequency in a range of 0 to 0.005 Hz, with approximately 0.001 Hz being a specific example, and a low-pass cutoff frequency in a range of 1 to 10 Hz, with approximately 5 Hz being a specific example.

Respiratory parameter generator 442 represents a specific embodiment of respiratory parameter generator 342 and produces one or more respiratory parameters using the respiratory signal. Such one or more respiratory parameters provide for monitoring of normal and abnormal respiratory activities. In various embodiments, the one or more respiratory parameters are indicative of one or more of respiratory rhythm, lung volume, and lung capacity. Examples of respiratory parameters indicative of respiratory rhythm include respiratory rate (respiratory cycle length), inspiratory period, expiratory period, and non-breathing period. Examples of respiratory parameters indicative of lung volume include tidal volume, inspiratory reserve volume, and expiratory reserve volume. Examples of respiratory parameters indicative of lung capacity include vital capacity, inspiratory capacity, and functional residual capacity.

Respiratory disorder detector 456 detects one or more respiratory disorders using the one or more respiratory parameters. Examples of respiratory disorders include central sleep apnea, obstructive sleep apnea, dyspnea, Cheyne-Stokes respiration, and asthma. In one embodiment, the one or more respiratory disorders are associated with heart failure.

Phrenic nerve activity detector 458 detects artificial stimulation of the phrenic nerve using the respiratory signal. The artificial stimulation of the phrenic nerve results in contractions of diaphragm 108. In one embodiment, delivery of cardiac pacing pulses unintentionally stimulates the phrenic nerve. Detection of diaphragmatic activities resulting from cardiac pacing allows for adjustment of pacing parameters to avoid the unintended stimulation of the phrenic nerve.

Pulmonary edema detector 460 detects pulmonary edema using the lymphatic sensor signal, where the lymphatic sensor signal is the transthoracic impedance signal. In one embodiment, pulmonary edema detector 460 detects pulmonary edema by monitoring a DC impedance, which includes a DC (and/or ultra-low-frequency) component of the transthoracic impedance signal that indicates lung fluid status. The pulmonary edema is an indication of acute decompensation in heart failure.

Posture monitor 450 produces a posture signal indicative of the posture of body 101 using the lymphatic sensor signal, where the lymphatic sensor signal is the accelerometer signal. Posture monitor 450 includes a band-pass filter to remove components of the respiratory signal that is not associated with the posture, such as the diaphragmatic activities. In one embodiment, the band-pass filter for producing the posture signal has a high-pass cutoff frequency in a range of 0 to 0.005 Hz, with approximately 0.001 Hz being a specific example, and a low-pass cutoff frequency in a range of 1 to 10 Hz, with approximately 5 Hz being a specific example.

Activity monitor 452 produces a gross activity signal indicative of the level of gross physical activity of body 101 using the lymphatic sensor signal, where the lymphatic sensor signal is the accelerometer signal. Activity monitor 452 includes a band-pass filter to remove components of the respiratory signal that is not associated with the gross physical activity, such as the diaphragmatic activities. In one embodiment, the band-pass filter for producing the gross activity signal has a high-pass cutoff frequency in a range of 0 to 0.005 Hz, with approximately 0.001 Hz being a specific example, and a low-pass cutoff frequency in a range of 1 to 10 Hz, with approximately 5 Hz being a specific example.

FIG. 5 is an illustration of an embodiment of a lymphatic sensor assembly 520 for placement in a lymphatic vessel such as thoracic duct 105. Lymphatic sensor assembly 520 represents an embodiment of lymphatic sensor assembly 320 and includes a lymphatic sensor 532 and a lymphatic sensor base 534. Lymphatic sensor 532 represents an embodiment of lymphatic sensor 332. Lymphatic sensor base 534 represents an embodiment of lymphatic sensor base 334. In one embodiment, lymphatic sensor assembly 520 represents an embodiment of lymphatic sensor assembly 120 and is incorporated into distal end 116 of lead 112. In another embodiment, lymphatic sensor assembly 520 represents an embodiment of lymphatic sensor assembly 220 and wirelessly communicates with another device via telemetry.

In the illustrated embodiment, lymphatic sensor base 534 is expandable. After being expanded, lymphatic sensor base 534 are in contact with the inner wall of thoracic duct 105, thereby restricting the movement of lymphatic sensor 532 in thoracic duct 105. In one embodiment, lymphatic sensor base 534 includes a stent that is expanded in the lymphatic vessel to maintain patency of the vessel. Lymphatic sensor 532 is incorporated into the stent. In another embodiment, lymphatic sensor base 534 includes a balloon. In an inflated state, the balloon is in contact with the inner wall of thoracic duct 105 to restrict the movement of lymphatic sensor 532 in thoracic duct 105. Lymphatic sensor 532 is incorporated onto the balloon. In a specific embodiment, the balloon has a tubular or other structure to limit the effect of lymphatic sensor assembly 520 on lymphatic flow through thoracic duct 105.

Lymphatic sensor base 534 is illustrated in FIG. 5 as a specific example of a fixation mechanism. In various embodiments, lymphatic sensor base 534 may include any form of fixation mechanism that restricts the movement of lymphatic sensor 532 in the vessel in which it is implanted.

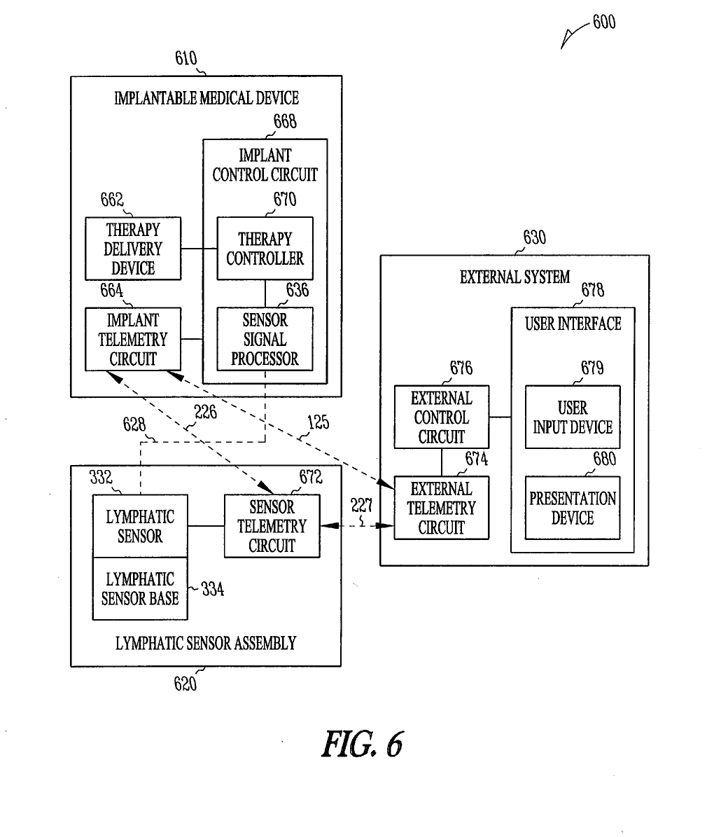

FIG. 6 is a block diagram illustrating an embodiment of a CRM system 600 into which respiratory monitoring system 100 or 200 is incorporated. CRM system 600 includes a lymphatic sensor assembly 620, an implantable medical device 610, and an external system 630.

Lymphatic sensor assembly 620 represents an embodiment of lymphatic sensor assembly 120, 220, 320, or 520 and includes lymphatic sensor 332 and lymphatic sensor base 334. As illustrated in FIG. 6, lymphatic sensor assembly 620 further includes a sensor telemetry circuit 672, if lymphatic sensor 332 communicates with implantable medical device 610 and/or external system 630 via telemetry (as in system 200). Sensor telemetry circuit 672 transmits the lymphatic sensor signal to implantable medical device 610 via telemetry link 226 and/or external system 630 via telemetry link 227. If lymphatic sensor 332 communicates with implantable medical device 610 through a wired link (as in system 100), conductors 628 incorporated into lead 112 provide the electrical connections.

Implantable medical device 610 represents an embodiment of implantable medical device 110 and includes an implant telemetry circuit 664, a therapy delivery device 662, and an implant control circuit 668. Implant telemetry circuit 664 allows implantable medical device 610 to communicate with external system 630 via telemetry link 125 and/or lymphatic sensor 332 via telemetry link 226. Therapy delivery device 662 delivers one or more therapies. Implant control circuit 668 includes a therapy controller 670 and a sensor signal processor 636. Sensor signal processor 636 represents an embodiment of sensor signal processor 336 or 436 and processes the lymphatic sensor signal sensed by lymphatic sensor 332. Therapy controller 670 controls the delivery of the one or more therapies using the processed lymphatic sensor signal.

External system 630 represents an embodiment of external system 130 and includes an external telemetry circuit 674, an external control circuit 676, and a user interface 678. External telemetry circuit 674 allows external system 630 to communicate with implantable medical device 610 via telemetry link 125 and/or lymphatic sensor 332 via telemetry link 227. External control circuit 676 controls the operation of external system 630. User interface 678 includes a user input device 679 and a presentation device 680. User input device 679 allows the physician or other caregiver to control the operation of CRM system 600, including the programming of implantable medical device 610 and/or lymphatic sensor 332. Presentation device 680 presents various information to the physician or other caregiver, such as signals acquired using implantable medical device 610 and/or lymphatic sensor 332 and information indicative of operational status of CRM system 600.

In the illustrated embodiment, implant control circuit 668 includes sensor signal processor 636. This allows, for example, implantable medical device 610 to control its therapeutic functions using the lymphatic sensor signal sensed by lymphatic sensor 332. In another embodiment, external control circuit 676 includes sensor signal processor 636. This allows, for example, the physician or other caregiver to monitor respiratory activities through user interface 678. In various embodiments, sensor signal processor 636 is distributed in implant control circuit 668 and/or external control circuit 676, depending on how the lymphatic sensor signal is used in CRM system 600.

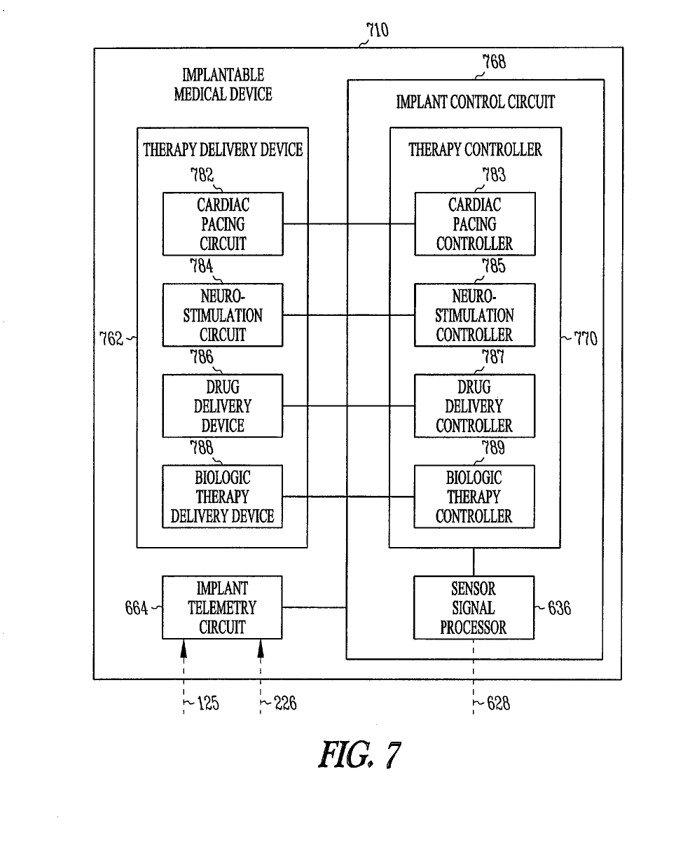

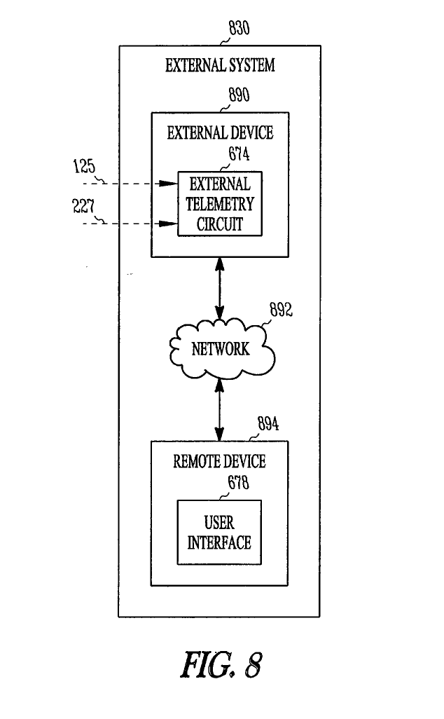

FIG. 7 is a block diagram illustrating an embodiment of an implantable medical device 710, which represents an embodiment of implantable medical device 610. Implantable medical device 710 includes a therapy delivery device 762, an implant control circuit 768, and implant telemetry circuit 664. Implant control circuit 768 represents a specific embodiment of implant control circuit 668 and includes a therapy controller 770 and sensor signal processor 636.

Therapy delivery device 762 represents a specific embodiment of therapy delivery device 662. Therapy controller 770 represents a specific embodiment of therapy controller 670. In the illustrated embodiment, therapy delivery device 762 includes a cardiac pacing circuit 782 that delivers pacing pulses, a neurostimulation circuit 784 that delivers neurostimulation, a drug delivery device 786 that delivers one or more drugs, and a biologic therapy delivery device 788 that delivers one or more biologic therapies. Therapy controller 770 includes a cardiac pacing controller 783 that controls the delivery of the pacing pulses, a neurostimulation controller 785 that controls the delivery of the neurostimulation, a drug delivery controller 787 that controls the delivery of the one or more drugs, and a biologic therapy controller 789 that controls the delivery of the one or more biologic therapies. In various embodiments, therapy delivery device 762 includes any one or more of cardiac pacing circuit 782, neurostimulation circuit 784, drug delivery device 786, and biologic therapy delivery device 788. Therapy controller 770 includes the corresponding one or more of cardiac pacing controller 783, neurostimulation controller 785, drug delivery controller 787, and biologic therapy controller 789.

In one embodiment, therapy controller 770 controls the delivery of the one or more therapies using the one or more respiratory parameters generated by respiratory parameter generator 342 or 442. For example, therapy controller 770 is a feedback controller that adjusts one or more therapy parameters using the one or more respiratory parameters as input. In another embodiment, therapy controller 770 controls the delivery of the one or more therapies using the respiratory signal produced by respiratory monitor 430 and one or more of the posture signal produced by posture monitor 450 and the gross activity signal produced by activity monitor 452. In one embodiment, cardiac pacing controller 783 adjusts pacing parameters using an outcome of the detection of the artificial stimulation of the phrenic nerve by phrenic nerve activity detector 458 to prevent the pacing pulses from activating the phrenic nerve. In one embodiment, cardiac pacing controller 783 adjusts pacing parameters using the respiratory rate produced by respiratory parameter generator 442, such that the heart rate changes according to the body's metabolic need. This provides, for example, physiologic control of the heart rate in response to physical activities in a patient with chronotropic incompetence. In one embodiment, neurostimulation controller 785 adjusts neurostimulation parameters to prevent the neurostimulation from producing intolerable effects on the respiratory activities. This provides, for example, prevention of neurostimulation from causing or worsening sleep disordered breathing in heart failure patients.

FIG. 8 is a block diagram illustrating an embodiment of an external system 830, which represents an embodiment of external system 630. As illustrated in FIG. 8, external system 830 is a patient management system including an external device 890, a telecommunication network 892, and a remote device 894. External device 890 is placed within the vicinity of implantable medical device 610 and includes external telemetry circuit 674 to communicate with implantable medical device 610 via telemetry link 125. Remote device 894 is in a remote location and communicates with external device 890 through network 892. Remote device 894 includes user interface 678 to allow the physician or other caregiver to monitor and treat a patient from a distant location and/or allowing access to various treatment resources from the remote location.

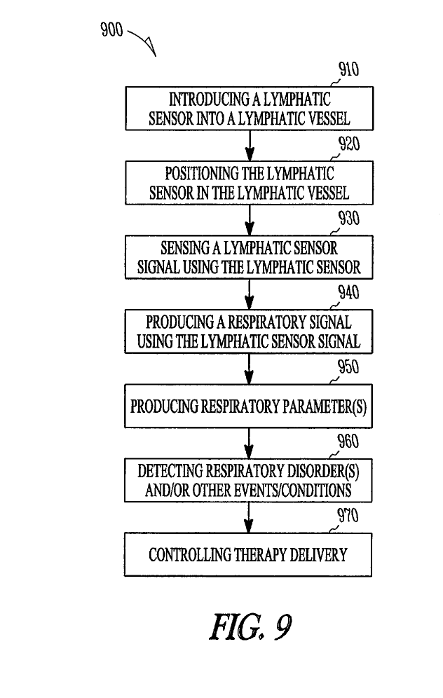

FIG. 9 is a flow chart illustrating a method 900 for monitoring respiratory activities using a sensor placed in a lymphatic vessel. In one embodiment, method 900 is performed in system 100, 200, or 600.

A lymphatic sensor is inserted into a lymphatic vessel of a patient at 910. The lymphatic sensor is a sensor that is implanted in a lymphatic vessel, or a location accessible through the lymphatic vessel, to sense a signal indicative of at least respiratory activities. In one embodiment, this lymphatic vessel is the thoracic duct. In one embodiment, the lymphatic sensor is incorporated into the distal end of an implantable transluminal lead having a proximal end configured for connection to an implantable medical device. To implant the lymphatic sensor into the thoracic duct, an opening is made on the subclavian vein, upstream from the junction of the subclavian vein and the ostium of the thoracic duct. The distal end of the lead is inserted into the subclavian vein through the opening and advanced toward the junction of the subclavian vein and the ostium of the thoracic duct downstream. Then, the lead is guided into the thoracic duct and advanced in the thoracic duct until the distal end reaches a region near the diaphragm. In one embodiment, the distal end of the lead is guided into a lymphatic vessel branching from the thoracic duct for placement in a region near the diaphragm. In another embodiment, the lymphatic sensor is a wireless device communicating with an implantable medical device via telemetry. The lymphatic sensor is implanted by being attached to the distal end of a percutaneous transluminal catheter. The process of sensor placement is substantially similar to that with the implantable transluminal lead, except that the catheter is withdrawn from the body after the lymphatic sensor is placed.

The lymphatic sensor is positioned in the lymphatic vessel, such as the thoracic duct or a vessel branching from the thoracic duct, at 920. In one embodiment, after the distal end of the lead or catheter reaches the region near the diaphragm, the lymphatic sensor is activated to sense a signal. The distal end of the lead or catheter is moved in the thoracic duct or the vessel branching from the thoracic duct until it reaches a position identified by satisfactory quality of the sensed signal. The lymphatic sensor is then stabilized in that position. In one embodiment, the lymphatic sensor is connected to a lymphatic sensor base with a fixation mechanism such as an expandable device that is expanded to contact the inner wall of the vessel to stabilize the lymphatic sensor in the vessel.

A lymphatic sensor signal is sensed using the lymphatic sensor at 930. The lymphatic sensor signal is indicative of respiratory activities. In one embodiment, the lymphatic sensor signal is a diaphragmatic activity signal indicative of movement of the diaphragm, such as an accelerometer signal or a stain gauge signal. In one embodiment, the accelerometer signal, if sensed, is also used for monitoring gross activity the body and/or posture of the body. In another embodiment, the lymphatic sensor signal is a transthoracic impedance signal indicative of pulmonary volume.

A respiratory signal indicative of the respiratory activities is produced using the lymphatic sensor signal at 940. In one embodiment, a gross activity signal indicative of a level of the gross physical activity of the body and/or a posture signal indicative of the posture of the body are also produced.

One or more respiratory parameters are produced using the respiratory signal at 950 to provide for monitoring of respiratory activities. The one or more respiratory parameters are indicative of one or more of respiratory rhythm, lung volume, and lung capacity. Examples of such respiratory parameters include respiratory rate (respiratory cycle length), inspiratory period, expiratory period, non-breathing period, tidal volume, inspiratory reserve volume, expiratory reserve volume, vital capacity, inspiratory capacity, and functional residual capacity.

One or more respiratory disorders and/or other events or conditions are detected using the one or more respiratory parameters at 960. Examples of the respiratory disorders include central sleep apnea, obstructive sleep apnea, dyspnea, Cheyne-Stokes respiration, and asthma. Examples of the other events or conditions include artificial stimulation of the phrenic nerve and pulmonary edema.

Delivery one or more therapies is controlled at 970. Examples of the therapies include cardiac pacing therapy, neurostimulation therapy, drug therapy, and biologic therapy. In one embodiment, the delivery of the one or more therapies is controlled using the respiratory signal (including the one or more respiratory parameters and detection of the respiratory disorder and/or the other events or conditions). In another embodiment, the delivery of the one or more therapies is controlled using the respiratory signal and one or more of the posture signal and the gross activity signal. In one embodiment, one or more parameters of the one or more therapies are adjusted using the one or more respiratory parameters as input for feedback control in a closed-loop system. In one embodiment, cardiac pacing parameters are adjusted using an outcome of the detection of the artificial stimulation of the phrenic nerve to prevent the pacing pulses from activating the phrenic nerve. In another embodiment, the cardiac pacing rate is adjusted using the respiratory rate such that the heart rate changes according to the body's metabolic need in case of chronotropic incompetence. In another embodiment, neurostimulation parameters are adjusted in response to the detection of a respiratory disorder to prevent the neurostimulation from producing intolerable effects on the respiratory activities.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for use in a body having a diaphragm and a lymphatic vessel, the system comprising:
a lymphatic sensor assembly configured to be implanted in the lymphatic vessel, the lymphatic sensor assembly including:
a lymphatic sensor configured to sense a lymphatic sensor signal indicative of respiratory activities; and
a lymphatic sensor base connected to the lymphatic sensor and configured to allow the lymphatic sensor assembly to be implanted in the lymphatic vessel; and
a sensor signal processor communicatively coupled to the lymphatic sensor, the sensor signal processor including:
a sensor signal input configured to receive the lymphatic sensor signal;
a respiration monitor coupled to the sensor signal input and configured to produce a respiratory signal indicative of the respiratory activities by processing the lymphatic sensor signal; and
a respiratory parameter generator coupled to the respiration monitor and configured to produce one or more respiratory parameters using the respiratory signal.

2. The system of claim 1, wherein the lymphatic sensor comprises a diaphragmatic activity sensor configured to sense a diaphragmatic activity signal indicative of movement of the diaphragm.

3. The system of claim 2, wherein the diaphragmatic activity sensor comprises an accelerometer configured to sense an accelerometer signal indicative of the movement of the diaphragm.

4. The system of claim 3, wherein the sensor signal processor comprises a posture monitor configured to produce a posture signal indicative of a posture of the body using the accelerometer signal.

5. The system of claim 3, wherein the sensor signal processor comprises an activity monitor configured to produce a gross activity signal indicative of a level of gross physical activity of the body using the accelerometer signal.

6. The system of claim 2, wherein the diaphragmatic activity sensor comprises a strain gauge configured to sense a strain gauge signal indicative of the movement of the diaphragm.

7. The system of claim 2, comprising an implantable medical device including the sensor signal processor and an implant telemetry circuit coupled to the sensor signal processor and configured to receive the diaphragmatic activity signal from the lymphatic sensor assembly via a wireless communication link, and wherein the lymphatic sensor assembly comprises a sensor telemetry circuit coupled to the diaphragmatic activity sensor and configured to transmit the sensed diaphragmatic activity signal to the implantable medical device via the wireless telemetry link.

8. The system of claim 1, wherein the lymphatic sensor comprises an impedance sensing electrode configured to sense a transthoracic impedance signal indicative of pulmonary volume.

9. The system of claim 8, wherein the sensor signal processor comprises a pulmonary edema detector configured to detect pulmonary edema using the transthoracic impedance signal.

10. The system of claim 1, wherein the lymphatic sensor base comprises a fixation mechanism configured to stabilize the lymphatic sensor assembly in the lymphatic vessel.

11. The system of claim 10, wherein the lymphatic sensor base comprises a stent configured to be placed in the lymphatic vessel.

12. The system of claim 10, wherein the lymphatic sensor base comprises a balloon configured to be placed in the lymphatic vessel.

13. The system of claim 1, comprising:
an implantable medical device including the sensor signal processor; and
an implantable transluminal lead including:
a proximal end configured to be connected to the implantable medical device;
a distal end; and
an elongate body coupled between the proximal end and a distal end, the elongate body having a distal portion connected to the distal end and configured to be placed in the lymphatic vessel,
wherein one or more of the distal end and the distal portion include the lymphatic sensor assembly.

14. The system of claim 1, comprising:
a therapy delivery device configured to deliver one or more therapies to the body; and
a therapy controller configured to control the delivery of one or more therapies using the one or more respiratory parameters.

15. The system of claim 14, wherein the therapy controller comprises a feedback controller adapted to adjust one or more therapy parameters of the one or more therapies using the one or more respiratory parameters as input.

16. The system of claim 14, wherein the sensor signal processor comprises a respiratory disorder detector configured to detect one or more respiratory disorders using the one or more respiratory parameters, and wherein the therapy controller is configured to adjust one or more therapy parameters of the one or more therapies in response to the detection of each of the one or more respiratory disorders.

17. A method for monitoring respiratory activities in a body having a diaphragm and a lymphatic vessel, the method comprising:
sensing a lymphatic sensor signal using a lymphatic sensor implanted in the lymphatic vessel, the lymphatic sensor signal indicative of respiratory activities;
producing a respiratory signal indicative of the respiratory activities using the lymphatic sensor signal; and
producing one or more respiratory parameters using the respiratory signal.

18. The method of claim 17, wherein sensing the lymphatic sensor signal using the lymphatic sensor comprises sensing a diaphragmatic activity signal indicative of movement of the diaphragm using an activity sensor.

19. The method of claim 18, wherein sensing the diaphragmatic activity signal indicative of movement of the diaphragm using the activity sensor comprises sensing an accelerometer signal indicative of the movement of the diaphragm using an accelerometer.

20. The method of claim 19, comprising producing one or more of an activity signal indicative of a level of gross physical activity of the body and a posture signal indicative of posture of the body using the accelerometer signal.

21. The method of claim 20, comprising:
delivering one or more therapies to the body; and
controlling delivery of the one or more therapies using the respiratory signal and the one or more of the activity signal and the posture signal.

22. The method of claim 17, wherein sensing the lymphatic sensor signal using the lymphatic sensor comprises sensing a transthoracic impedance signal indicative of pulmonary volume using an impedance sensing electrode.

23. The method of claim 17, comprising:
delivering one or more therapies to the body;
controlling delivery of the one or more therapies using one or more therapy parameters; and
adjusting the one or more therapy parameters using the one or more respiratory parameters.

24. The method of claim 23, comprising detecting one or more respiratory disorders using the one or more respiratory parameters, and wherein adjusting the one or more therapy parameters comprises adjusting the one or more therapy parameters in response to the detection of each of the one or more respiratory disorders.

25. The method of claim 24, wherein delivering the one or more therapies comprises delivering neurostimulation, and adjusting the one or more therapy parameters comprises adjusting one or more neurostimulation parameters in response to the detection of each of the one or more respiratory disorders.

26. The method of claim 23, wherein delivering the one or more therapies comprises delivering cardiac pacing pulses, and adjusting the one or more therapy parameters comprises adjusting one or more pacing parameters.

27. The method of claim 26, comprising detecting artificial stimulation of the phrenic nerve using the respiratory signal, and wherein adjusting the one or more pacing parameters comprises adjusting the one or more pacing parameters in response to the detection of the artificial stimulation of the phrenic nerve to prevent the cardiac pacing pulses from activating the phrenic nerve.

28. The method of claim 26, wherein producing the one or more respiratory parameters comprises producing a respiratory rate, and wherein adjusting the one or more pacing parameters comprises adjusting the one or more pacing parameters using the respiratory rate.

29. The method of claim 23, wherein delivering the one or more therapies comprises delivering one or more of a cardiac pacing therapy, a neurostimulation therapy, a drug therapy, and a biologic therapy.
